(19) United States
(12) Patent Application Publication
(10) Pub. No.: US 2023/0310936 A1
(43) Pub. Date: Oct. 5, 2023

(54) METHOD AND VARIOUS APPLICATIONS OF DELIVERING OBJECT RELATED MESSAGES BY USING WIDE-FREQUENCY SOUND SIGNAL

(71) Applicants: Chia-Hung Chen, New Taipei (TW); Chia-Chi Su, New Taipei (TW); Jian-Long Su, New Taipei (TW)

(72) Inventors: Chia-Hung Chen, New Taipei (TW); Chia-Chi Su, New Taipei (TW); Jian-Long Su, New Taipei (TW)

(21) Appl. No.: 18/023,075

(22) PCT Filed: Aug. 30, 2021
(86) PCT No.: PCT/CN2021/115348

§ 371 Date: Feb. 24, 2023

Related U.S. Application Data
(60) Provisional application No. 63/072,215, filed on Aug. 30, 2020.
(60) Provisional application No. 63/075,205, filed on Sep. 6, 2020.

(51) Int. Cl. A63B 24/00; H04B 11/00

(52) U.S. Cl. CPC A63B 24/0062; H04B 11/00; A63B 22/02

(57) ABSTRACT

Method of delivering the object related message by using the format of the wide-frequency sound signal is proposed. Due to both the well-known Doppler effect and the well-known phenomena that the intensity is inversely proportional to the distance in the three-dimensional space, the transmitted wide-frequency sound signal itself indicates some properties of the objected attached by the wide-frequency sound signal transmitter, such as the motion and the distance. Besides, the delivered wide-frequency sound signal also carries the contents detected by one or more corresponding detectors. Such method has many possible applications, such as intelligent fitness equipment, health care, indusial application and so on.

### FIELD OF THE INVENTION

The present invention is related to a method of delivering the object related message by using the wide-frequency sound signal, and also is related to various applications of the wide-frequency sound signal transmitter/receiver.

### BACKGROUND OF THE INVENTION

During the past years, many types of sensors have been developed to detect various properties of various kinds of objects. For example, the gyroscopes used to detect the direction, the accelerometer used to detect the acceleration force, the multi-axis sensors used to detect a multiple of forces and moments simultaneously, the motion sensor used to detect the nearby object' motion, the barcode sticker used to present the existence of the object, the piezoelectric used detect the touch and the relative motion between objects, the magnetometers used to detect the magnetic object, the proximity sensor used to detect the nearby object, and so on. Besides, in general, these sensors are embedded with Wi-Fi, Bluetooth or any other wireless chips, even with cable lines, to deliver the detected object related messages.

However, the usage of the wireless chips and/or the cable lines meets unavoidably some disadvantages. For example, the number of the wireless connection channels is finite. In general, a commercial product, such as a mobile phone, usually has only single-digit wireless channels and/or single-digit cable ports, and then it is very difficult to use a commercial product to receive the messages sent from a number of sensors simultaneously. For example, the power consumption required to build up and maintain the wireless communication is high and the hardware cost to setup the cable communication is high, also both the power consumption and the cost is scaled up with the increase in the number of objects to be detected and/or the number of object properties to be detected is increased. For example, the spatial flexibility of the capable lines is low, also the effective transmission distance of the wireless communication is limited.

Therefore, the conventional skills almost only receive messages sent from a limited number of detectors at the same time, also both the detector and the communication (no matter wireless communication and/or cable line) are required to detect the object property and to deliver the object related message respectively. Reasonably, at least the hardware cost, the operation complexity, and the available flexibility of different applications are all limited.

Accordingly, new method for delivering object related message(s) and various applications of the new method are desired.

### SUMMARY OF THE INVENTION

The proposed invention uses the wide-frequency sound signal transmitter, such as the audio signal transmitter and/or the ultrasonic signal transmitter, to replace the wireless communication and the cable used by the conventional skills. Therefore, by analyzing the contents carried by the wide-frequency sound signal, even by analyzing the spectrum, the waveform and/or the spectrogram of the wide-frequency sound signal itself, one or more properties of a variable number of objects are handled effectively and simultaneously.

The invention proposes a method using at least one wide-frequency sound signal emitter and one wide-frequency sound signal receiver to deliver one or more object related messages, especially to deliver multiple wide-frequency sound signals contains such object related messages simultaneously. Each wide-frequency sound signal emitter is attached to or connected to an object, or viewed as fixed at a certain part of an object. Each wide-frequency sound signal emitter is connected to one or more detectors correspond to the same object. The wide-frequency sound signal receiver is a device capable of receiving the audio signal and/or the ultrasonic signal. Indeed, the invention does not limit the details of both the wide-frequency sound signal receiver and the wide-frequency sound signal receiver. Any existed, on-developed or to-be-appeared device is optional.

Apparently, by using different wide-frequency sound signal transmitters, different wide-frequency sound signals are transmitted with different frequencies, even different intensities, respectively at the same time. Therefore, a wide-frequency sound signal receiver, such as the microphone embedded with a smartphone, simply distinguish each of these wide-frequency sound signals sent from different wide-frequency sound signal transmitter from others. Moreover, a wide-frequency sound signal receiver receives numerous wide-frequency sound signals at the same time because it just receives all sound signals sent from the surrounding environment. After that, all received sound signals are analyzed to identify what each individual sound signal indicates. Reasonably, by using the wide-frequency sound signal as the medium to carry the object related message, a number of objects are monitored individually at the same time. Particularly, the upper limitation of the number of the wide-frequency sound signals to be received by a commercial mobile product, such as smart phone with embedded microphone, is apparently high than the number of the wireless communication channels owned by a commercial mobile product, such as smartphone or pad. Many ways are optional to increase simply the upper limitation. For example, increase the frequency range of the sound signal that the microphone receiver configured to receive, and increase the resolution rate of the microphone for sounds signals with different frequencies.

Furthermore, the wide-frequency sound signal itself indicates some messages of the objected attached by the wide-frequency sound signal transmitter. In contrast, both the wireless communication and the cable only indicate the object related messages through the contents carried by wireless signals and/or wire signals. One key factor is the well-known Doppler effect that the change in frequency of a wave in relation to an observer who is moving relative to the wave source. Hence, both the relative movement direction and the relative movement speed between a wide-frequency sound signal transmitter and a wide-frequency sound signal receiver is simply monitored by analyzing the wide-frequency sound signal transmitted by the transmitter and then received by the receiver. In other words, the motion properties of an object are simply monitored by attaching a such transmitter to it without further using any other detector(s). Another key factor is the well-known phenomenon that the intensity of sound signal in three-dimensional space is inversely proportional to the square of the relative distance. Hence, not only the variation of the relative distance between a wide-frequency sound signal transmitter and a wide-frequency sound signal receiver is decided by analyzing the variation of the intensity of the received sound signal, but also the relative distance between the transmitter and the receiver is decided by analyzing the intensity of the received sound signal if the intensity of the transmitted sound signal is known. In other words, the position properties of an object are simply monitored by attaching a such transmitter to it without further using any other detector(s).

In addition, the invention need not to limit what is connected to the wide-frequency sound signal receiver and is configured to analyze the received wide-frequency sound signal. Any processer, any artificial intelligence, any sound-signal library, application software, and/or any circuits is optional. Surely, one more option of the invention is to connect both the wide-frequency sound signal transmitted and the detector together with the object and then to deliver the object related message via the transmitted wide-frequency sound signal.

One application of such method is the intelligent fitness equipment. The messages related to the motion status of a user and/or the mechanical status of a fitness equipment are transmitted at the format of the wide-frequency sound signals to the movable device, such as a mobile device, such that the messages is further analyzed to evaluate the how the user exercises. By using the wide-frequency sound signal transmitter/receiver, a single user optionally uses a mobile device simultaneously to evaluate his/her exercise status through the wide-frequency sound signal received by the embedded microphone in the mobile device and to operate other peripheral device(s) through the finite wireless communication channels owned by the mobile device. Even, in a spinning bike room or in a weight training room, a number of spinning bike or a number of weight training devices communicate with one and only one host, such as a laptop, through the wide-frequency sound signal. Thus, the placement of the host is flexible without the limitation of the cable, also one and only one host simultaneously monitor a large number of bikes/devices that is obvious larger than the number of the wireless communication channels owned by the host.

One application of such method is the health care. The messages related to the body posture of a user are transmitted at the format of the wide-frequency sound signals to movable device, such as a mobile device, such that the messages are further analyzed to evaluate the how the user's body posture. For example, by simply attach a wide-frequency sound signal transmitter on the chest of a patient or a person in motion, the ups and downs of the chest caused by breathing is easily presented by the variations of the frequency and/or the intensity of the transmitted wide-frequency sound signals. Hence, by analyzing the received signals, it is intuitive to measure the breathing frequencies and depth. Then, the health professional monitor simply the patient' respiratory status, such as anesthesia condition, obstructive sleep apnea, cough, asthma and so on. Such as a nurse works in a crowd ward simply just uses one table to monitor a number of patients' respiratory statuses. Also, the person in motion simply checks the respiratory status for the exercise loading or performance. Similarly, by attaching one or more wide-frequency sound signal transmitters on one patient's hands and legs, how accurately the patient control the motion of the limbs also is quantitatively detected.

One application of such method is the industrial application of a combination of a pressure detector and a wide-frequency sound signal transmitter, where the detected pressure is transmitted at the format of the wide-frequency sound signal. The change of the pressure reflects the change of liquid stored in a tank when the combination is placed under the tank, and then such combination is useful to detect the liquid level. A similar application is the weight determination when the combination is placed under a pan.

### BRIEF DESCRIPTION OF THE DRAWINGS

Other advantages, objectives and features of the present invention will become apparent from the following description referring to the attached drawings.

FIG. 1A and FIG. 1B are two essential flowcharts of the method of delivering the object related messages by using the wide-frequency sound signal.

FIG. 2A and FIG. 2B present two examples of the relation between audio intensity and distance change and the relation between audio frequency and distance respectively.

FIG. 3A to FIG. 3D schematically illustrate some examples of the applications of the proposed invention.

FIG. 4A to FIG. 4B schematically illustrate two examples of the applications of the proposed invention.

FIG. 5 is an essential flowchart of some examples as a method of monitoring human body.

### DETAILED DESCRIPTION OF THE INVENTION

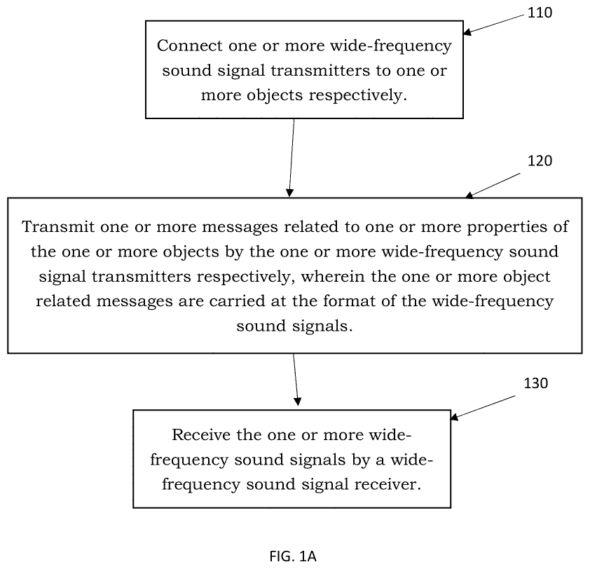

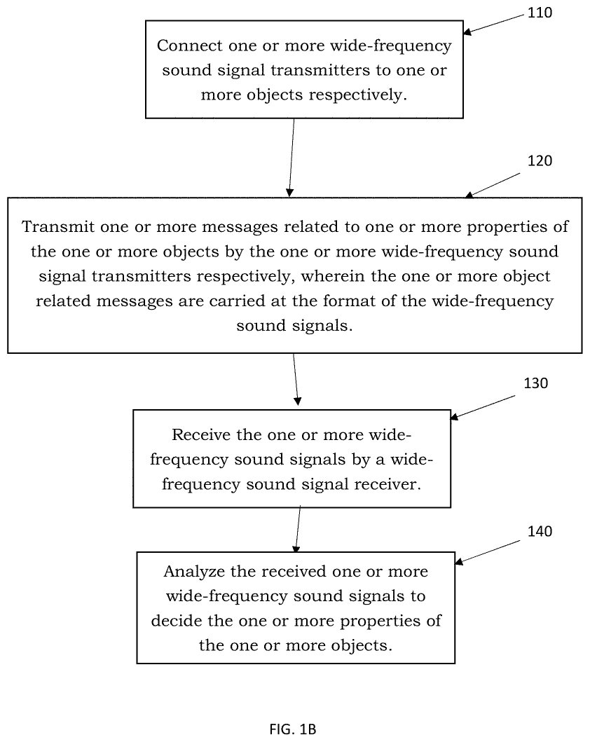

Some embodiments are related to a method of delivering the object related message by using the wide-frequency sound signal. Two essential flowcharts of such method are shown in FIG. 1A and FIG. 1B respectively. Initially, as shown in block 110, connect one or more wide-frequency sound signal transmitters to one or more objects respectively. Next, as shown in block 120, transmit one or more messages related to one or more properties of the one or more objects by the one or more wide-frequency sound signal transmitters respectively, wherein the one or more object related messages are carried at the format of the wide-frequency sound signals. Then, as shown in block 130, receive the one or more wide-frequency sound signals by a wide-frequency sound signal receiver. Besides, optionally, as shown in block 140, analyze the received one or more wide-frequency sound signals to decide the one or more properties of the one or more objects. FIG. 1A focuses on the delivery of the wide-frequency sound signal, and then may be viewed as the flowchart of any equipment just configured to replace the usage of wireless communication and/or cable communication by using the wide-frequency sound signal. FIG. 1B further includes the step of analyzing the received wide-frequency sound signal, and then may be viewed as the flowchart of any equipment configured to deliver and acquire the object related message(s) by using the wide-frequency sound signal.

Apparently, one main feature is the usage of the wide-frequency sound signal, no matter audio signal and/or ultrasonic signal, wherein each messages related to an object is carried at the format of the wide-frequency sound signal. In contrast, the conventional skills use the cable and/or the wireless communication, such as Bluetooth or Wi-Fi, to deliver the object related messages. In particular, because there are already many commercial products for audio signal and due to many commercial audio and/or ultrasonic signal transmitters and receivers, the proposed invention needs not to handle related hardware and software but directly uses any available product. Especially, while only audio signal and/or ultrasonic signal has to be generated and transmitted, these commercial products usually have the benefits of low cost and low power consumption. That is to say, the present invention is realized simply by using any existing commercial products.

One essential benefit of the proposed invention is that many commercial products are capable of receiving the wide-frequency sound signal, such as a microphone embedded in a mobile device, a microphone embedded in a host, or a recent Hi-Res ADC MEMS MIC. Hence, by using a commercial device, a user can communicate with one or more wide-frequency sound signal transmitters via the wide-frequency sound signals and also with one or more peripheral devices via the cable and/or the wireless communications simultaneously. That is to say, the finite number of the cable and/or the wireless communication channels of a commercial product does not limit how many wide-frequency sound signal transmitters is connected, or viewed as does not limit how many objects are monitored by the usage of the wide-frequency sound signal transmitter/receiver. In other words, optionally, some variations of the proposed invention use one and only one wide-frequency sound signal receiver to communicate with and multiple wide-frequency sound signal transmitters.

Another essential benefit of the proposed invention is that one or more variations of a wide-frequency sound signal itself indicate one or more properties of an object attached by a wide-frequency sound signal transmitter. The well-known Doppler effect discloses that the change in frequency of a sound is related to the relative motion between a sound source and a sound an observer. Therefore, the relative distance between a wide-frequency sound signal receiver and one or more wide-frequency sound signal receivers are detectable by analyzing the variation of the frequency of each wide-frequency sound signal received by the receiver respectively. Also a well-known phenomenon discloses that the intensity of a wave is inversely proportional to the relative distance in the three-dimensional space. Therefore, the change of the relative distance between a wide-frequency sound signal receiver and one or more wide-frequency sound signal receivers are detectable by analyzing the variation of the intensity of each wide-frequency sound signal received by the receiver respectively. Even, if the default signal intensity of one or more certain wide-frequency sound signal transmitters are known, the practical relative distances between the wide-frequency sound signal receiver and one or more certain wide-frequency sound signal transmitters are detectable by analyzing the practical intensity of each corresponding wide-frequency sound signal received by the receiver respectively.

The synergy of the above two advantages is very apparent. By using the wide-frequency sound signal to deliver the object relate message, the wireless communication and/or the wired communication of a commercial device is not interfered, a large number of objects connected to a number of such transmitters are monitored at the same time, even both relative motion and relative distance between such receiver and some objects attached by such transmitters are detectable simultaneously.

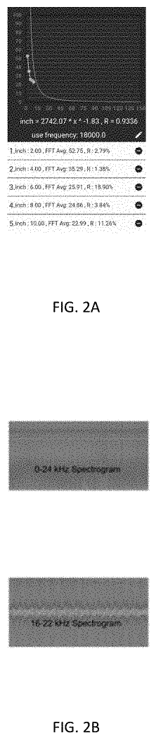

Only as an auxiliary information, FIG. 2A and FIG. 2B present two examples of the relation between audio intensity and distance change and the relation between audio frequency and distance respectively. In the former, the wide-frequency sound signal transmitter is operated in 18 KHz, and the differential distances between the sound source and a smartphone with embedded microphone are 2, 4, 6, 8, and 10 inches. Clearly, the fast Fourier transform (FFT) array values are related to the sound signal intensity, but are more linear than traditional dB units. Clearly, the FFT array values (audio intensity) and the algorithm are high regression (R2 is 0.9336) and validated, and higher performance such transmitter is provided longer distance determination between such transmitter and such receiver with differential frequencies. In the latter, the distance moving is between 0 to 10 inches, the moving rate is around 0˜30 m/sec, and the transmitter is operated in 18 KHz. Based on the Doppler shift, the frequencies are between 16552 to 19725 Hz, and the spectrograms are presented as two graphics separated. Clearly, the background noise does not affect the signals transmitted and analyzed, and a real-time audio library is used easily to determine the moving rate and counting the moving and status. The higher frequency indicates that they are closing to each other, and the lower frequency indicates that they are separating from each other. Clearly, by using an artificial intelligence with trained algorithm, the speed and the moving status with the period of the spectrogram is determined simply.

Surely, when a wide-frequency sound signal receiver is capable of communicating with two of more wide-frequency sound signal transmitters, different communications with different transmitters has to be distinguished. Hence, one option is to configure at least two wide-frequency sound signal transmitters such that different wide-frequency sound signals are transmitted with different signal frequencies, and then the wide-frequency sound signal receiver distinguish simply by their respective frequencies. Another option is to configure at least two wide-frequency sound signal transmitters such that different wide-frequency sound signals are transmitted with different signal intensities, although this option is somehow not as efficient as the above option. Therefore, it is desired to configure one or more wide-frequency sound signal transmitters respectively. Moreover, a commercial product popularly allows the built-in microphone to receive a signal with a large frequency range but the required frequency of a transmitted wide-frequency sound signal for carrying the object related messages usually is limited. Hence, one more option is to configure at least one wide-frequency sound signal transmitter such that the frequency of the transmitted wide-frequency sound signal is adjustable, such as to be adjusted to be different than the frequency of another transmitted wide-frequency sound signal. And another option is to configure at least one wide-frequency sound signal transmitter such that the intensity of the transmitted wide-frequency sound signal is adjustable.

Furthermore, in addition to analyze the variation of frequency and/or intensity of the wide-frequency sound signal itself for monitor the relative motion and/or relative distance, the invention optionally analyzes the contents contained in such signal for monitor other property(s). In such option, one or more detectors are connected to the one or more objects respectively, wherein at least one object is connected to a wide-frequency sound signal transmitter and one or more detectors at the same time. In this way, one or more contents of one or more object properties detected by one or more detectors are transmitted at the format of the wide-frequency sound signal.

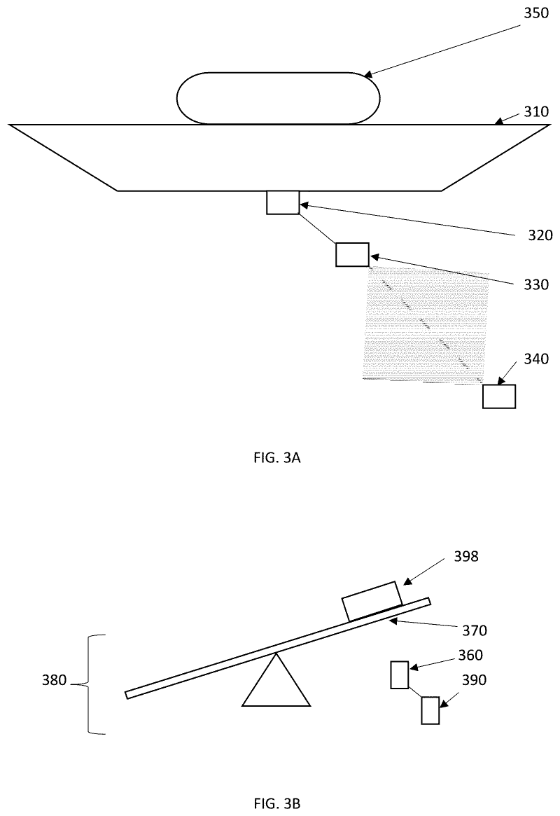

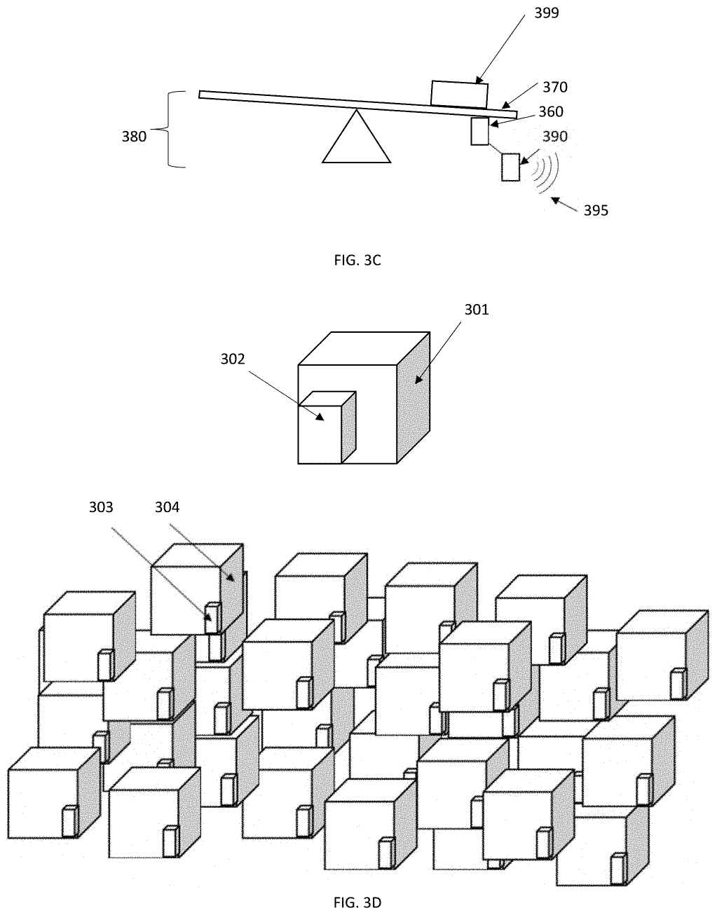

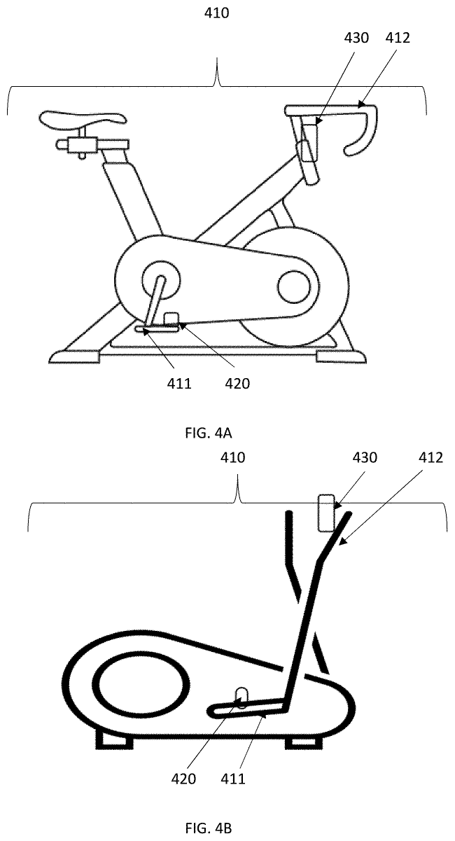

Without question, depending on the details of the used detector(s), different object properties are detected and transmitted. Hence, the proposed methods correspond to numerous applications. For example, as shown in FIG. 3A, one application is the object weight measurement. A pressure detector 320 is positioned under a weight pan 320 to detect the weight of any goods 350 placed on the weight pan 320 and connected to the wide-frequency sound signal transmitter 330. The measured weight message is transmitted at the format of the wide-frequency sound signal and from the wide-frequency sound signal transmitter 330 to the wide-frequency sound signal receiver 340, wherein further analyzing is optional. For example, as shown in FIG. 3B and FIG. 3C, another application also is the object weight measurement. A touch detector 360 is placed under and separated from the suspended terminal 370 of the seesaw-like device 380 and connected to a wide-frequency sound signal transmitter 390. A warning message is not carried by a transmitted wide-frequency sound signal 395 when the weight of the object 398 placed on the suspended terminal 370 does not exceed a critical value so that the suspended terminal 370 is still separated from the touch detector 360, but is carried by a transmitted wide-frequency sound signal 395 when the weight of the object 399 placed on the suspended terminal 370 exceeds a critical value so that the suspended terminal 370 falls and touches the touch detector 360. For example, as shown in FIG. 3D, one more application is the gym management. One and only one host 301 with at least one embedded wide-frequency sound signal receiver 302 is configured to receive at least one wide-frequency sound signal transmitted from at least one wide-frequency sound signal transmitter 303 embedded in at least one fitness equipment 304 respectively. In this way, when different such transmitters 303 are adjusted to be operated in different frequency range, the messages related to the status of all fitness equipment 304 are delivered between the signal host 301 and these such transmitters 303. Optionally, different fitness equipment 304 are connected to different detectors respectively, such as more message related to these fitness equipment 304 are detectable. Hence, the signal host 301 grasp the status of many fitness equipment 304 at the same time, and is not limited by the channel number of the wireless communication (such as Wi-Fi/Bluetooth) and/or the wire communication (such as cable) owned by the host 301. Significantly, it increases the convenience of gym management. Just as an example, in a spinning bike classroom with dozens of spinning bikes, an instructor uses his mobile phone to simultaneously grasp the respective speed of each spinning bike when dozens of such transmitter are attached to the wheel of these spinning bikes respectively or to simultaneously grasp the respective load weight and climbing angle when dozens of such transmitters are connected to the controller of these spinning bikes respectively.

Some embodiments are related to an intelligent fitness equipment having a fitness equipment and a wide-frequency sound signal transmitter connected to a portion of the fitness equipment. Till now, the well-known commercial intelligent fitness equipment uses popularly Bluetooth, Wi-Fi and/or cable to deliver the message detected by the detector placed on a portion of the fitness equipment. For example, the intelligent fitness equipment provided by some big companies, such as Decathlon, Alatech, Bryton and Garmin. Thus, a user of a commercial intelligent fitness equipment handles easily his motion status by using his mobile phone or tablet. Anyway, as discussed above, the number of wireless communication channels of a mobile phone is quite limited. Thus, it is often difficult to connect the detect for receiving signals related his motion status and to connect other periphery device for processing other actions simultaneously, such as to connect both Bluetooth speakers and Wi-Fi projector to enjoy multimedia program while exercising. Significantly, in these embodiments, the usage of the wide-frequency sound signal transmitter overcome such difficulty while the microphone of the user's mobile device performs the function of the wide-frequency sound signal receiver.

These embodiments have a unique advantage. The used wide-frequency sound signal transmitter itself is capable of detecting the operating status of the intelligent fitness equipment. As discussed above, due to the well-known Doppler effect and the well-known inverse square phenomena, the change of frequency and/or intensity of the delivered wide-frequency sound signal is related to related motion and/or related distance between an intelligent fitness equipment (where a wide-frequency sound signal transmitter is attached) and a mobile device owned by the user (wherein a built-in microphone acts as a wide-frequency sound signal receiver is built-in). Hence, these proposed intelligent fitness equipment uses only a set of wide-frequency sound signal transmitter/receiver to monitor the motion and/or position of a fitness equipment with efficiency and low cost. In contrast, the well-known commercial intelligent fitness equipment uses both a detector to detect the operating status of this intelligent fitness equipment and a wireless/wired communication channel to deliver the detected messages. For example, the Hall element is commonly attached to the pedal of the spinning bike so as to record the number of pedal rotations. For example, the gyroscope and/or the multi-axis sensor is commonly used to monitor dynamically the change of motion of a fitness equipment. For example, the video detector is commonly used to recode images and sounds during the operation period of a fitness equipment. For example, the motion sensor is commonly used to track the operating status of a fitness equipment. Clearly, no matter what kind of detector is used by the well-known commercial intelligent equipment, a wireless communication device and/or a wired communication device is further required to delivered the messages detected by the used detector.

Furthermore, the advantage of high efficiency and low cost is more apparent due to most motions correspond to periodic limb movements, especially due to most indoor motions corresponds to movement back and forth with a certain distance along a fixed direction. Hence, all required messages is fully grasped by using the combination of the wide-frequency sound signal transmitter and the wide-frequency sound signal receiver. No other message is required and has to be collected by using other detector.

Accordingly, in some related embodiments, such as the intelligent spinning bike and the intelligent rowing machine, the fitness equipment has at least a stable assembly and a movable assembly. Where, the wide-frequency sound signal transmitter is attached to the movable assembly so as to transmit the wide-frequency sound signal corresponding to the motion status of the movable assembly. Of course, in some other related embodiments, such as the intelligent dumbbell, it is formed in one piece and the wide-frequency sound signal transmitter is attached to the one-piece structure so as to transmit the wide-frequency sound signal corresponding to the motion status of the whole one-piece structure.

In addition, to enhance the user convenience, the fitness equipment has optionally a receiving terminal. Hence, a wide-frequency sound signal receiver, or viewed as the user's mobile device, is optionally placed on the receiving terminal while the user uses the device during the period of using the proposed intelligent fitness equipment. Anyway, if only considering the usage of such wide-frequency sound signal, whether the user place the wide-frequency sound signal receiver, or viewed as the user's mobile device, on the fitness equipment or outside the fitness equipment is not the most essential factor of the proposed intelligent fitness equipment.

In addition, how to attach the wide-frequency sound signal transmitter to the fitness equipment is not limited. Optionally, the wide-frequency sound signal transmitter is embedded in a dongle with a battery set and a fixing element. One advantage is the commercial wide-frequency sound signal transmitter has lower power consumption, so the proposed intelligent fitness equipment can work for a long time without needing to replace the barratry. The details of the fixing elements also are not limit. Some commercial options include magnetic buttons, 3M back sticker, strap and packaging bag.

The operation frequency of the wide-frequency sound signal transmitter is not limited. These embodiments only require that the transmitted wide-frequency sound signal has enough bandwidth to carry message and its' propagation is not interfered clearly between such transmitter and such receiver. Optionally, some conducted experiments use the following frequency range: 19.2 KHz and 20 KHz, or even the following frequency range: 14˜22 KHz and 24˜48 KHz. Optionally, some conducted experiments use the following frequency range: below 20 KHz, 20˜48 KHz, over 48 KHZ and any combination thereof. Optionally, some conducted experiments use the following range: frequency range of audio signal, frequency range of ultrasonic signal for general life applications, frequency range of ultrasonic signal for industrial applications and any combination thereof. Anyway, other frequency also is acceptable for the proposed intelligent fitness equipment.

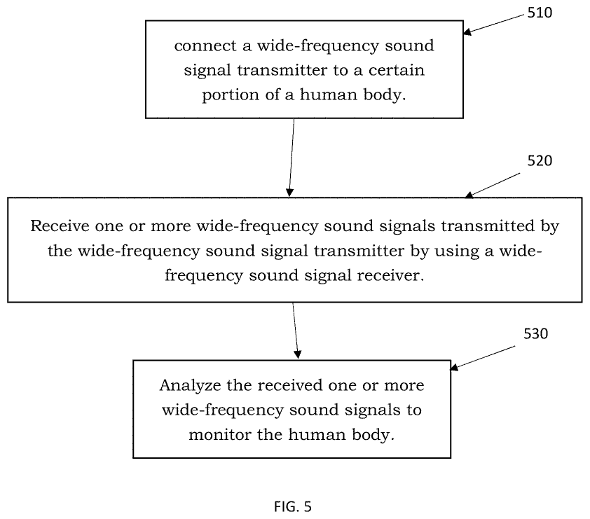

As shown in FIG. 4A, one example is that the fitness equipment 410 is a spinning bike (or a bicycle) and the wide-frequency sound signal transmitter 420 is attached to the pedal 411 of the spinning bike, wherein the wide-frequency sound signal receiver 430 is the mobile phone placed closed to the hand holder 412 of the fitness equipment 410. Clearly, the rotation rate of the pedal 411 is equivalent to the relative movement rate between the pedal 411 and the hand holder 412, and the Doppler shift of the wide-frequency sound signal received by the receiver 430 quantitatively present such rate. As shown in FIG. 4B, another example is that the fitness equipment 410 is an elliptical trainer and the wide-frequency sound signal transmitter 420 is attached to the pedal 411 of the elliptical trainer, wherein the wide-frequency sound signal receiver 430 is the mobile phone placed closed to the hand holder 412 of the fitness equipment 410. Clearly, the repeated displacement of the pedal 411 is equivalent to the repeated change of the relative distance between the pedal 411 and the hand holder 412, and the variation of the intensity of the wide-frequency sound signal received by the receiver 430 quantitatively present such rate. Although not particularly illustrated in figures, there are numerous examples. For example, the fitness equipment is a rowing machine and the wide-frequency sound signal transmitter is attached to an oar grip. Hence, the back and forth movement of the oar grip is presented as the Doppler shift of the wide-frequency sound signal received by a microphone separated away from the oar grip. For example, the fitness equipment is a treadmill and the wide-frequency sound signal transmitter is attached to a track. The functional relationship between such transmitter and a receiver placed outside the fitness equipment is similar to the functional relationship between the wide-frequency sound signal transmitter 420 and the wide-frequency sound signal receiver 430 shown in FIG. 4A. Two more examples are that the fitness equipment is a walking machine and a wide-frequency sound signal transmitter is attached to a pedal and that the fitness equipment is a walking machine and the wide-frequency sound signal transmitter is attached to a grip. Still two more examples are that the fitness equipment is a dumbbell and the wide-frequency sound signal transmitter is attached to a certain terminal of the dumbbell and that the fitness equipment is a dumbbell and the wide-frequency sound signal transmitter is attached to a handle used to connecting one or more levels. Obviously, the proposed intelligent fitness equipment does not limit the details of the used fitness equipment, the only requirement is that the wide-frequency sound signal is attached to a portion of the fitness equipment such that the motion and the position of such transmitter is closely equal to that of the fitness equipment.

Some embodiments are related to a method of monitoring human body. The essential flowchart of such method is shown in FIG. 5. Initially, as shown in block 510, connect a wide-frequency sound signal transmitter to a certain portion of a human body. Then, as shown in block 520, receive one or more wide-frequency sound signals transmitted by the wide-frequency sound signal transmitter by using a wide-frequency sound signal receiver. After that, as shown in block 530, analyze the received one or more wide-frequency sound signals to monitor the human body. Reasonably, by attaching the wide-frequency sound signal transmitter to the certain portion of the human body, it is effective to monitor the motion of the human body, at least the movement of the certain portion of the human body, by analyzing the variation of the received wide-frequency sound signal itself, even by analyzing the received wide-frequency sound signal itself. Reasonably, by further connecting a detector to the human body and the wide-frequency sound signal transmitter, one or more human body related message, such as the blood pressure and the body temperature, are delivered at the format of the wide-frequency sound signal and then received and analyzed simply.

One application of the proposed method is the respiration measurement. By attaching a wide-frequency sound signal transmitter to the chest of a human body, such as chest of a patient, the motion and the movement of the wide-frequency sound signal transmitter is highly equivalent to the up and down variation of the chest during the respiration process. Hence, the transmitted wide-frequency sound signal itself is useful information for deciding both the respiration rate and the breathing intensity of the human body. Then, by analyzing the received one or more wide-frequency sound signals received by a wide-frequency sound signal receiver, an effective respiration measurement is achieved without using any complex and/or expensive respirometer.

Another application of the proposed method is the human body posture monitoring. By attaching one or more wide-frequency sound signal transmitters to one or more portions of the human body, the human body is monitored continuously by analyzing continuously the received one or more wide-frequency sound signals. For example, when a patient's body posture is changed between laying down, sitting up and standing up, the positions of one or more such transmitters placed on the patient's hands, feet, shoulders and/or abdomens also are changed correspondingly. Therefore, by using one and only one such receiver at fixed position to continuously receive the signals from different such transmitters, whether the patient has obviously changed his body posture is simply determined. For example, when a user is doing yoga in a yoga classroom, the information about the extent degree of the user's limbs is tracked by using multiple such transmitters placed at the ends of the user's limbs and one such receiver (such as a computer host placed in the yoga classroom). For example, when a user is practicing boxing, two wide-frequency sound signal transmitters operated at different frequency ranges are place each of his gloves respectively and a wide-frequency sound signal receiver connected to an analyzing device capable of analyzing the received wide-frequency sound signals is fixedly placed a certain distance in front of the user. Hence, not only the punch rate of the user is determined by detecting the number of Doppler shifts inversions in the received wide-frequency sound signals over a period of time, but also the punch attack distance of the user is determined by detecting the intensity change degree of the received wide-frequency sound signals over a period of time.

Still another application of the proposed method is the medical monitoring. By a medical detector to the human body and connecting the wide-frequency sound signal transmitter to the detector, one or more medical messages are sent at the format of the wide-frequency sound signal. Then, the communication between the wide-frequency sound signal transmitter and the wide-frequency sound signal receiver may carry the medial message related to the human body. For example, by connecting multiple wide-frequency sound signal transmitters to multiple electrocardiographs respectively, the heartbeat status messages of a number of monitored patients detected by these electrocardiographs are delivered to a wide-frequency sound signal receiver at the format of wide-frequency sound signal together. Hence, in a ward with a large number of inpatients, a nurse on duty simply track the heartbeat status of these inpatients in real time through both the wide-frequency sound signal and processor built inside a handheld device.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A method of delivering the object related message by using the wide-frequency sound signal, comprising:
connecting one or more wide-frequency sound signal transmitters to one or more objects respectively;
transmitting one or more messages related to one or more properties of the one or more objects by the one or more wide-frequency sound signal transmitters respectively, wherein the one or more object related messages are carried at the format of the wide-frequency sound signals; and
receiving the one or more wide-frequency sound signals by a wide-frequency sound signal receiver.

2. The method according to claim 1, further comprising using one and only one wide-frequency sound signal receiver and a plurality of wide-frequency sound signal transmitters.

3. The method according to claim 1, wherein the wide-frequency sound signal is chosen from a group consisting of the following: an audio signal and an ultrasonic signal.

4. The method according to claim 1, further comprising at least one of the following:
configuring at least two wide-frequency sound signal transmitters such that different wide-frequency sound signals are transmitted with different signal frequencies;
configuring at least two wide-frequency sound signal transmitters such that different wide-frequency sound signals are transmitted with different signal intensities;
configuring at least one wide-frequency sound signal transmitter such that the frequency of the transmitted wide-frequency sound signal is adjustable; and
configuring at least one wide-frequency sound signal transmitter such that the intensity of the transmitted wide-frequency sound signal is adjustable.

5. The method according to claim 1, further comprising analyzing the received one or more wide-frequency sound signals to decide the one or more properties of the one or more objects.

6. The method according to claim 5, further comprising deciding the relative motion between a certain wide-frequency sound signal transmitter and the wide-frequency sound receiver by analyzing the frequency variation of the wide-frequency sound signal transmitted therebetween by reference to the Doppler effect.

7. The method according to claim 5, further comprising at least one of the following:
deciding the relative distance variation between a certain wide-frequency sound signal transmitter and the wide-frequency sound signal receiver by analyzing the intensity variation of the wide-frequency sound signal transmitted therebetween by reference to the phenomena that the intensity is inversely proportional to the relative distance in the three-dimensional space; and
deciding the relative distance between a certain wide-frequency sound signal transmitter and the wide-frequency sound signal receiver by analyzing the intensity variation of the wide-frequency sound signal transmitted therebetween by reference to the phenomena that the intensity is inversely proportional to the relative distance in the three-dimensional space if the default signal intensity of the certain wide-frequency sound signal transmitter is known.

8. The method according to claim 1, further comprising connecting one or more detectors to the one or more objects respectively, wherein at least one object is connected to a wide-frequency sound signal transmitter and one or more detectors at the same time, wherein one or more contents of one or more object properties detected by one or more detectors are transmitted at the format of the wide-frequency sound signal.

9. The method according to claim 8, further comprising at least one of the following:
placing a pressure detector positioned under a weight pan to detect the weight of any goods placed on the weight pan, and transmitting a wide-frequency sound signal carrying the weight message; and
placing a touch detector under and separated from a suspended terminal of a seesaw-like device, such that a warning message is carried by a transmitted wide-frequency sound signal when the weight placed on the suspended terminal exceeds a critical value so that the suspend terminals falls and touches the touch detector.

10. The method according to claim 1, further comprising using one and only one host with at least one embedded wide-frequency sound signal receiver to receive at least one wide-frequency sound signal transmitted from at least one wide-frequency sound signal transmitter embedded in at least one fitness equipment respectively.

11. An intelligent fitness equipment, comprising:
a fitness equipment; and
a wide-frequency sound signal transmitter, wherein the wide-frequency sound signal transmitter is connected to a portion of the fitness equipment.

12. The intelligent fitness equipment according to claim 11, wherein the fitness equipment has at least a stable assembly and a movable assembly, and wherein the wide-frequency sound signal transmitter is attached to the movable assembly.

13. The intelligent fitness equipment according to claim 11, wherein the fitness equipment has a receiving terminal, wherein a wide-frequency sound signal receiver is placed on the receiving terminal.

14. The intelligent fitness equipment according to claim 11, further comprising at least one of the following:
the wide-frequency sound signal transmitter is embedded in a dongle with a battery set and a fixing element; and
the operation frequency of the wide-frequency sound signal transmitter is chosen from a group consisting of the following: below 20 KHz, 20-48 KHz, over 48 KHz and any combination thereof.

15. The intelligent fitness equipment according to claim 11, further comprising at least one of the following:
the fitness equipment is a spinning bike and the wide-frequency sound signal transmitter is attached to a pedal;
the fitness equipment is a rowing machine and the wide-frequency sound signal transmitter is attached to an oar grip;
the fitness equipment is a treadmill and the wide-frequency sound signal transmitter is attached to a track;
the fitness equipment is a walking machine and the wide-frequency sound signal transmitter is attached to a pedal; and
the fitness equipment is a walking machine and the wide-frequency sound signal transmitter is attached to a grip.

16. The intelligent fitness equipment according to claim 11, further comprising at least one of the following:
the fitness equipment is a dumbbell and the wide-frequency sound signal transmitter is attached to a certain terminal of the dumbbell;
the fitness equipment is a dumbbell and the wide-frequency sound signal transmitter is attached to a handle used to connecting one or more levels.

17. A method of monitoring human body;
connecting a wide-frequency sound signal transmitter to a certain portion of a human body;
receiving one or more wide-frequency sound signals transmitted by the wide-frequency sound signal transmitter by using a wide-frequency sound signal receiver; and
analyzing the received one or more wide-frequency sound signals to monitor the human body.

18. The method according to claim 17, further comprising attaching the wide-frequency sound signal transmitter to the chest of the human body such that both the respiration rate and the breathing intensity are monitored by analyzing the received one or more wide-frequency sound signals to decide the motion and the movement of the wide-frequency sound signal transmitter.

19. The method according to claim 17, further comprising attaching one or more wide-frequency sound signal transmitters to one or more portions of the human body, so as to monitor continuously the human body by analyzing continuously the received one or more wide-frequency sound signals.

20. The method according to claim 17, further comprising attaching a medical detector to the human body and connecting the wide-frequency sound signal transmitter to the detector such that one or more medical messages are sent by the wide-frequency sound signal transmitter at the format of the wide-frequency sound signal.
